(19) United States
(12) Patent Application Publication
(10) Pub. No.: US 2024/0156501 A1
(43) Pub. Date: May 16, 2024

(54) Anchor

(71) Applicant: Blue Sky Technologies, LLC, Louisville, KY (US)

(72) Inventor: Frank Castro, Louisville, KY (US)

(21) Appl. No.: 18/518,673

(22) Filed: Nov. 24, 2023

Related U.S. Application Data
(62) Division of application No. 17/843,963, filed on Jun. 18, 2022.
(60) Provisional application No. 63/222,132, filed on Jul. 15, 2021.
(60) Provisional application No. 63/222,115, filed on Jul. 15, 2021.

(51) Int. Cl. A61B 17/84; A61B 17/70

(52) U.S. Cl. CPC A61B 17/844; A61B 17/7032; A61B 17/7035; A61B 2017/00871

(57) ABSTRACT

An anchor including shape memory metals, shape memory polymers or a combination thereof. The anchor has surgical and nonsurgical uses.

### PRIORITY

In view of the Mar. 15, 2023 Restriction Requirement, this Application is a Division of Application 17843963—Anchor—filed Jun. 18, 2022 that claimed the benefit of US Provisional Application No. 63222132—Bone Fastener Device—filed Jul. 15, 2021 and US Provisional Application No. 63222115—Anchor—filed Jul. 15, 2021. Please examine claims 10-19.

### BACKGROUND OF THE INVENTION

### A. Field of the Invention

Among other things, select preferred embodiments of the present invention are an anchor for bone. The current implant is particularly suited for implantation or use in mammalian spinal or other boney tissues. Select embodiments of the current invention include surface treatments in anticipation of improving attachment of bone to the anchor.

Among other things, select preferred embodiments of the present invention are nonsurgical anchors. The current anchor is particularly suited for insertion into a ceiling, floor, roof or wall of a structure.

### B. Description of the Previous Art

Any discussion of references cited in this Description of the Previous Art merely summarizes the disclosures of the cited references and Applicant makes no admission that any cited reference or portion thereof is relevant prior art. Applicant reserves the right to challenge the accuracy, relevancy and veracity of the cited references.

References that may indicate a state-of-the-art for the current invention include: 1) US Published Patent Application 20170100163-Palmer et al. discloses devices for controlling the unloading of superelastic and shape memory orthopedic implants; 2) U.S. Pat. No. 9,539,039-Schaller et al. discloses humeral head fixation device for osteoporotic bone; 3) US Published Patent Application 20040230193-Cheung et al. discloses a fixation device; 4) US Published Patent Application 20180078293-Hustedt et al. discloses an implant and method for long bone fixation; and 5) U.S. Pat. No. 9,439,770-Biedermann et al. discloses an implant for stabilizing vertebrae or bones.

Among other things, none of the above listed references disclose:

An anchor adapted for implantation into a mammal and interconnection with an apparatus distinct from the anchor; the anchor comprising a shaft comprising: a) a first lengthwise end comprising a conical penetrating edge adapted for insertion into and through a surgically created opening in a bone; b) a second lengthwise end comprising a non-penetrating margin positioned opposite the conical penetrating edge; c) a first section proximate the first lengthwise end and a second section proximate the second lengthwise end, wherein the first and second sections are rigid constructions; and d) a flexible mesh comprising: i) a first circumference connected to an inward side of the first lengthwise end and a second circumference connected to an inward side of the second lengthwise end; and ii) a plurality of interwoven shape memory wires or threads or a combination thereof connected between the first circumference and the second circumference; the plurality of interwoven shape memory wires or threads creating apertures ranging in size from about 1 micrometer to about 5 millimeters; the sizes of the apertures adapted to improve passage of the flexible mesh through the opening, wherein prior to insertion through the opening, a breadth of a middle cross-section of the flexible mesh is equal to or less than diameters of the inward side of the first lengthwise end and the inward side of the second lengthwise end, and subsequent to insertion through the opening, the middle cross-section of the flexible mesh expands to at least twice a size of a bore of the opening, thereby preventing pullout of the anchor from the spinal bone.

An anchor adapted for implantation into a mammal and interconnection with an apparatus distinct from the anchor; the anchor comprising: a) a shaft comprising: i) a first lengthwise end comprising a first segment including two divisions; ii) a second lengthwise end comprising a rigid second segment and a non-penetrating margin positioned opposite the first segment; the rigid second segment connected to the first segment; and iii) the two divisions comprising a temperature sensitive composition, wherein the temperature sensitive composition's is a first contour until a transition temperature for the temperature sensitive composition is achieved, thereby transforming the temperature sensitive composition into a second contour.

An anchor insertable into a ceiling, floor, roof or wall of a structure; the anchor comprising a shaft comprising: a) a first lengthwise end comprising a penetrating edge adapted to penetrate the ceiling, floor, roof or wall of the structure; b) a second lengthwise end comprising a non-penetrating margin positioned opposite the penetrating edge; c) a first section proximate the first lengthwise end and a second section proximate the second lengthwise end, wherein the first and second sections are rigid constructions; and d) an intermediate section positioned between the first section and the second section; the intermediate section comprising a temperature sensitive composition, wherein the temperature sensitive composition is a first contour until a transition temperature for the temperature sensitive composition is achieved, thereby transforming the temperature sensitive composition into a second contour.

An anchor insertable into a ceiling, floor, roof or wall of a structure; the anchor comprising a shaft comprising: a) a first lengthwise end comprising a first segment; the first segment adapted to extend through the ceiling, floor, roof or wall of the structure; b) a second lengthwise end comprising a non-penetrating margin positioned opposite the first lengthwise end and a second segment connected with the first segment; and c) a plurality of divisions of first segment comprising a temperature sensitive composition, wherein the temperature sensitive composition's is a first contour until a transition temperature for the temperature sensitive composition is achieved, thereby transforming the temperature sensitive composition into a second contour.

### SUMMARY OF THE INVENTION

The present invention provides a biocompatible bone fastener or anchor that can be interlocked with an apparatus distinct from the implant. Preferred embodiments are provided with shafts including one or more rigid sections or segments. Associated with the shafts are sections or segments that include a temperature sensitive composition.

Within the scope of the current invention and depending on the materials selected for the temperature sensitive composition, the transition temperature ranges for the change from a first distinct shape to a second distinct shape of the composition can range from about −20 degrees Celsius to about +110 degrees Celsius. By way of illustration, the temperature sensitive compositions of the current anchor can have a first contour prior to achieving the transition temperature and a second contour after achieving the transition temperature. Depending on the transition metals incorporated into the temperature sensitive composition, the range for transition of the current temperature sensitive composition from a first contour to a second contour can range from about −20 degrees Celsius to about +40 degrees Celsius. Select preferred embodiments of the current invention can have transition temperature of from about 20 degrees Celsius to about 33 degrees Celsius. Still other preferred embodiments can have a transition temperature of less than about 35 degrees Celsius. Along with shape memory alloys and shape memory polymers, preferred embodiments of the anchor can be provided with surface treatments in anticipation of improving attachment of bone to the anchor.

With regard to spinal surgical procedures, prior art traditional fixation screws fixation stability is dependent on the composition of both cortical and cancellous bone. Pullout strength is directly correlated with the total volume of bone between the screw's threads. Cortical bone provides superior holding power because it is denser than cancellous bone. Those skilled in the art recognize an individual's cortical bone is always 20 to about 100 times stronger than his/her cancellous bone. Further, those skilled in the art also know that increasing the screw length for osteoporotic bone rarely provides satisfactory resistance against the fixation screw from pulling-out or backing-out of bone.

Because of its novel structure and temperature sensitive composition, the present bone fastener or anchor has greater resistance against pulling-out or backing-out of osteoporotic bone. It is believed that the current invention's resistance to pull-out or back-out improves implant-construct stability, higher bone fusion rates and better postoperative clinical outcomes than prior art fixation screws. For surgical procedures involving bone, the current anchor can be inserted through a small incision into the bone tissue. Once inside the bone, the anchor changes contour to improve contact area with the inside surface of the cortical bone when compared to the contact area of a traditional screw. It is believed that a three millimeter expansion of the current anchor will improve the pullout strength 60 to 300 times compared to a typical screw with a 1 mm thread pitch

An aspect of the present invention is to provide an anchor with a penetrating edge.

Still another aspect of the present invention is to provide an anchor with a shaft including one or more rigid sections or segments.

It is still another aspect of the present invention is to provide a bone fastener or anchor incorporating a temperature sensitive composition adapted for a first contour at a first temperature and a second contour at a second temperature.

Still another aspect of the present invention is to provide an anchor wherein each temperature sensitive segment has a transition temperature where the segment is a first contour at or about a first transition temperature and second contour at or about the transition temperature.

It is still another aspect of the present invention to provide temperature sensitive composition that is a default configuration about the transition temperature of the temperature sensitive composition and a second temporary configuration when the temperature sensitive composition is cooled below the transition temperature.

Yet still another aspect of the present invention is to provide an anchor with greater resistance against pulling-out or backing-out of osteoporotic bone than current available fixation screws.

Still another aspect of the present invention is to provide an anchor adapted for connection with an apparatus distinct from the anchor.

It is still another aspect of the present invention to provide a bone fastener or anchor capable of improving resistance to pull-out or back-out.

Yet still another aspect of the present invention is to an anchor capable of improving construct stability, higher bone fusion rates and better postoperative clinical outcomes than prior art fixation screws.

A preferred embodiment of the current invention can be described an anchor adapted for interconnection with an apparatus distinct from the anchor; the anchor comprising a shaft comprising: a) a first lengthwise end comprising a conical penetrating edge adapted for insertion into and through a surgically created opening in a bone; b) a second lengthwise end comprising a non-penetrating margin positioned opposite the conical penetrating edge; c) a first section proximate the first lengthwise end and a second section proximate the second lengthwise end, wherein the first and second sections are rigid constructions; and d) a flexible mesh comprising: i) a first circumference connected to an inward side of the first lengthwise end and a second circumference connected to an inward side of the second lengthwise end; and ii) a plurality of interwoven shape memory wires or threads or a combination thereof connected between the first circumference and the second circumference; the plurality of interwoven shape memory wires or threads creating apertures ranging in size from about 1 micrometer to about 5 millimeters; the sizes of the apertures adapted to improve passage of the flexible mesh through the opening, wherein prior to insertion through the opening, a breadth of a middle cross-section of the flexible mesh is equal to or less than diameters of the inward side of the first lengthwise end and the inward side of the second lengthwise end, and subsequent to insertion through the opening, the middle cross-section of the flexible mesh expands to at least twice a size of a bore of the opening, thereby preventing pullout of the anchor from the spinal bone.

Another preferred embodiment of the current invention can be described as an anchor adapted for interconnection with an apparatus distinct from the anchor; the anchor comprising: a) a shaft comprising: i) a first lengthwise end comprising a first segment including two divisions; ii) a second lengthwise end comprising a rigid second segment and a non-penetrating margin positioned opposite the first segment; the rigid second segment connected to the first segment; and iii) the two divisions comprising a temperature sensitive composition, wherein the temperature sensitive composition's is a first contour until a transition temperature for the temperature sensitive composition is achieved, thereby transforming the temperature sensitive composition into a second contour.

The anchor can inserted into a ceiling, floor, roof or wall of a structure. Depending on the composition into which the anchor will be inserted, the current invention can be self-tapping. It is believed that the current anchor can support a load of up to about 35 kilograms when the anchor is inserted into a generally vertical surface and a load of up to about 9 kilograms when the anchor is inserted into a generally horizontal surface.

An aspect of the present invention is to provide an anchor with a penetrating edge.

Still another aspect of the present invention is to provide an anchor with a shaft including one or more rigid sections or segments.

It is still another aspect of the present invention is to provide an anchor incorporating a temperature sensitive composition that becomes a first contour at a first temperature and a second contour at a second temperature.

Still another aspect of the present invention is to provide an anchor wherein each temperature sensitive section or segment has a transition temperature where the section or segment is a first contour at or about a first transition temperature and second contour at or about the transition temperature.

It is still another aspect of the present invention to provide temperature sensitive composition that has a default configuration about the transition temperature of the temperature sensitive composition and a second temporary configuration when the temperature sensitive composition is cooled below the transition temperature.

Yet still another aspect of the present invention is to provide an anchor with greater resistance against pulling-out or backing-out than currently available anchors.

Still another aspect of the present invention is to provide an anchor adapted for connection with an apparatus distinct from the anchor.

It is still another aspect of the present invention is to provide an anchor that can be cooled to its transition temperature with commonly available resources such as ice, freezers and liquefied gases.

A preferred embodiment of the current invention can be described as an anchor insertable into a ceiling, floor, roof or wall of a structure; the anchor comprising a shaft comprising: a) a first lengthwise end comprising a penetrating edge adapted to penetrate the ceiling, floor, roof or wall of the structure; b) a second lengthwise end comprising a non-penetrating margin positioned opposite the penetrating edge; c) a first section proximate the first lengthwise end and a second section proximate the second lengthwise end, wherein the first and second sections are rigid constructions; and d) an intermediate section positioned between the first section and the second section; the intermediate section comprising a temperature sensitive composition, wherein the temperature sensitive composition is a first contour until a transition temperature for the temperature sensitive composition is achieved, thereby transforming the temperature sensitive composition into a second contour.

Another preferred embodiment of the current invention can be described as an anchor insertable into a ceiling, floor, roof or wall of a structure; the anchor comprising a shaft comprising: a) a first lengthwise end comprising a first segment; the first segment adapted to extend through the ceiling, floor, roof or wall of the structure; b) a second lengthwise end comprising a non-penetrating margin positioned opposite the first lengthwise end and a second segment connected with the first segment; and c) a plurality of divisions of first segment comprising a temperature sensitive composition, wherein the temperature sensitive composition's is a first contour until a transition temperature for the temperature sensitive composition is achieved, thereby transforming the temperature sensitive composition into a second contour.

It is the novel and unique interaction of these simple elements which creates the apparatus and methods, within the ambit of the present invention. Pursuant to Title 35 of the United States Code and the Articles of the Patent Cooperation Treaty, descriptions of preferred embodiments follow. However, it is to be understood that the best mode descriptions do not limit the scope of the present invention.

### BRIEF DESCRIPTION OF THE DRAWINGS

FIG. 1 is a perspective of a first preferred embodiment of the anchor.

FIG. 2 is a perspective of a first preferred embodiment of the anchor.

FIG. 3 is a perspective of a second preferred embodiment of the anchor.

FIG. 4 is a perspective of a second preferred embodiment of the anchor.

FIG. 5 is a perspective of a third preferred embodiment of the anchor.

FIG. 6 is a perspective of a third preferred embodiment of the anchor.

FIG. 7 is a perspective of a fourth preferred embodiment of the anchor.

FIG. 8 is a perspective of a fourth preferred embodiment of the anchor.

FIG. 9 is a perspective of a fifth preferred embodiment of the anchor.

FIG. 10 is a perspective of a fifth preferred embodiment of the anchor.

FIG. 11 is a perspective of a sixth preferred embodiment of the anchor.

FIG. 12 is a perspective of a sixth preferred embodiment of the anchor.

FIG. 13 is a perspective of a seventh preferred embodiment of the anchor.

FIG. 14 is a perspective of a seventh preferred embodiment of the anchor.

FIG. 15 is a perspective of an eighth preferred embodiment of the anchor.

FIG. 16 is a perspective of an eighth preferred embodiment of the anchor.

FIG. 17 includes perspectives of configurations of heads usable with the anchor.

### DESCRIPTION OF THE PREFERRED EMBODIMENTS

Although the disclosure hereof is detailed to enable those skilled in the art to practice the invention, the embodiments published herein merely exemplify the present invention.

The Surgical Preferred Embodiments

In the most general sense, the present invention is a bone fastener or anchor for implantation into a surgically created cavity where the anchor is adapted for connection with an apparatus distinct from the anchor. Among other things, the current invention can be adapted for use with vertebra or other bone tissues. The present anchor is particularly adapted for use in the cervical region of the spine. Polymethymethacrylate is an adhesive particularly well suited for use with the current anchor.

Preferred embodiments of the present invention are manufactured of titanium alloys, stainless steel, shape memory alloys, non-resorbable polymers or any other composition acceptable in the art. Within the scope of the present invention, it has advantageously been discovered that shafts (40F, 40S) can have lengths from about 2 to about 10 millimeters; polyaxial heads (150) can have lengths of from about 5 millimeters to about 25 millimeters; sockets (154) of polyaxial heads (150) can have depths from about 3 millimeters to about 23 millimeters, diameters from about 4 millimeters to about 20 millimeters, lateral openings widths (162, 164) from about 3 millimeters to about 10 millimeters.

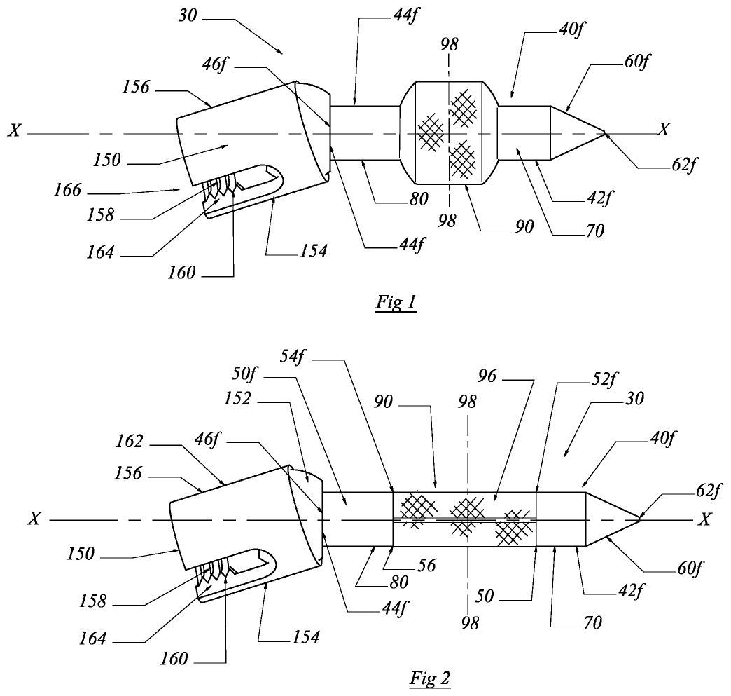

FIGS. 1 and 2 are perspectives of a first preferred embodiment of anchor (30). Within the scope of the current invention, anchor (30) can be interconnected with an apparatus distinct from the implant. Among other things, it has been discovered that anchor (30) is useful for spinal surgeries, and, in particular, surgeries for the posterior cervical region of the spine.

A preferred embodiment of anchor (30), enabled in FIGS. 1 and 2, includes shaft (400 and polyaxial head (150). Shaft (400 includes first lengthwise end (420 that is provided with penetrating edge (600. In select preferred embodiments, penetrating edge (600 converges to point (620 creating a conical penetrating edge (600. Opposite conical penetrating edge (600 of anchor (30) is non-penetrating margin (460 of second lengthwise end (440 of shaft (400.

Shaft (400 includes first section (70), second section (80) and intermediate section (90) positioned between first section (70) and second section (80). First section (70) is proximate first lengthwise end (420 and second section (80) is proximate second lengthwise end (440. Within the scope of the current invention, first section (70) and second section (80) are constructed from rigid biocompatible materials.

Intermediate section (90) of shaft (400 is positioned between first section (70) and second section (80). Intermediate section (90) is constructed of a temperature sensitive composition that can include shape memory metals, shape memory polymers or a combination thereof. These shape memory materials have a first contour above the transition temperature and a second configuration below the transition temperature of the shape memory materials. By way of illustration, when intermediate section or mesh (90) is cooled below the transition temperature, the second temporary contour of anchor (30) allows surgical insertion of anchor (30) into the patient. After placement into the patient, when anchor (30) warms above the transition temperature, anchor (30) returns to the first contour.

In select preferred embodiments, intermediate section (90) is constructed as a mesh. Intermediate section (90) can include one or more shape memory alloys or metals, shape memory polymers or a combination thereof. In some preferred embodiments, the shape memory alloy is a biocompatible alloy of nickel and titanium with percentages of nickel selected as a matter of predetermined medical engineering parameters. Within the scope of the present invention, shape memory polymers can include polyurethanes, epoxies, polyolefins or polyesters.

As shown in FIG. 1, intermediate flexible mesh (90) is expanded and is above its transition temperature, e.g., room temperature. A plurality of interwoven shape memory wires (96) or polymer fibers (96) or a combination thereof creates the flexible mesh (90) with apertures ranging in size from about 1 micrometer to about 5 millimeters. Flexible mesh (90) is connected to the first circumference (520 of inward side (50) of the first lengthwise end (420 and the second circumference (540 of the inward side (56) of the second lengthwise end (440. Prior to expansion, flexible mesh (90) is equal to or less than the diameters of the inward side (50) of the first lengthwise end (420 and the inward side (56) of the second lengthwise end (440. Unexpanded flexible mesh (90) can be passed through a surgically created opening in a bone. Subsequent to passage through the surgically created opening, the middle cross-section (98) of the flexible mesh (90) expands to a size that prevents extraction of anchor (30) through the surgical opening. Depending on the physical properties of the materials selected to manufacture mesh (90), the breadth of middle cross-section (98) of the flexible mesh (90) can expand two or more times the size of the bore of the surgical opening.

As shown in FIG. 2, intermediate section or mesh (90) was cooled below its transition temperature resulting in a second unexpanded contour for intermediate section or mesh (90). Generally, second contour of anchor (30) can be utilized by the surgeon for insertion into bone. For example, first section (70) and intermediate section (90) can be inserted in the lateral mass of the posterior cervical spine. After implantation, when intermediate section (90) is warmed above the transition temperature, intermediate section (90) returns to its first expanded configuration. When intermediate section (90) returns to its first configuration inside the lateral mass, intermediate section (90) can assist in preventing back out or pull out of anchor (30) from the surgically created cavity. In operation, this surgical configuration can clamp a greater volume of cortical bone between expanded intermediate section or mesh (90) and polyaxial head (150) of anchor (30) than is possible with current lateral mass fixation screws.

In select preferred embodiments, polyaxial head (150) is connected to non-penetrating margin (460 of shaft (400. Polyaxial head (150) of anchor (30) is adapted for connection with an apparatus (not shown) distinct from anchor (30). Examples of apparatus connectable to polyaxial head (150) include but are not limited to: rods, bars, cross-links, screws and locking nuts. Polyaxial head (150) is provided with spheroid (152) connected to non-penetrating margin (460 of second lengthwise end (440 of shaft (400. Prior to surgical fixation, the combination of spheroid (152) and socket (154) allows polyaxial head (150) to be moved in a multitude of axes relative to the longitudinal axis (X-X) of shaft (400. Socket (154) is provided with an outward housing (156) and inward receptacle (158) including one or more threads (160). Selected preferred embodiments of housing (156) can be provided with openings (162, 164, 166) adapted to receive one or more apparatus distinct from anchor (30).

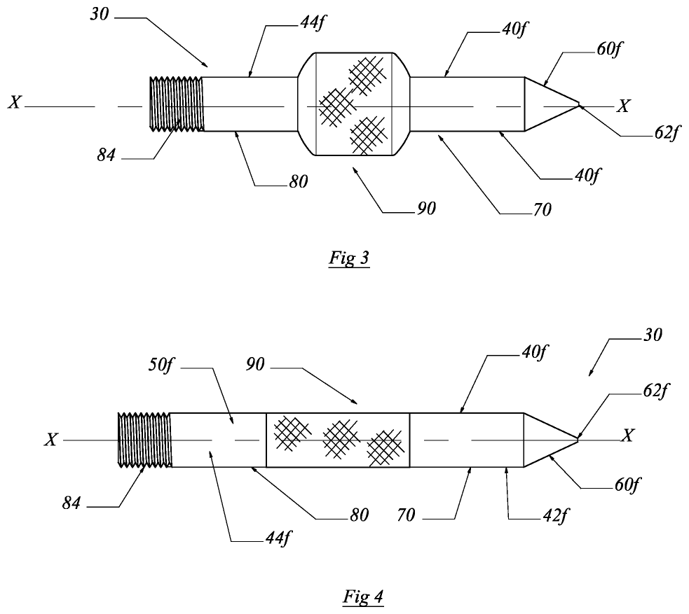

The embodiments, enabled in FIGS. 3 and 4, function similarly to the embodiments represented by FIGS. 1 and 2. However, the embodiments of FIGS. 3 and 4 embodiments do not utilize polyaxial head (150). Instead, second section (80) of shaft (400 is provided with at least one thread (84) for connection with an apparatus distinct from anchor (30). Thread (84) can engage an apparatus distinct from anchor (30) such as a nut (not shown).

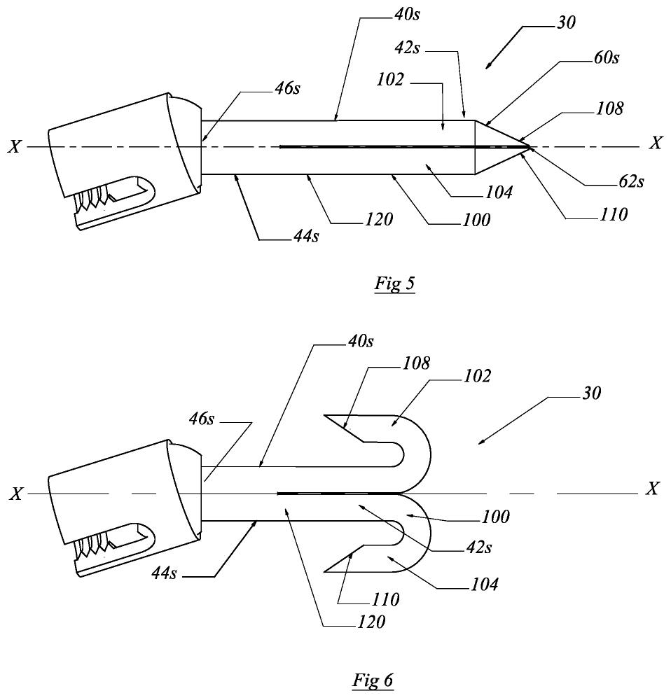

Among other things, another preferred embodiment of anchor (30), enabled in FIGS. 5 and 6, includes shaft (40s) and polyaxial head (150). Shaft (40s) includes first lengthwise end (42s) that is provided with penetrating edge (60s). In select preferred embodiments, penetrating edge (60s) converges to point (62s). Opposite penetrating edge (60s) of anchor (30) is non-penetrating margin (46s) of second lengthwise end (44s) of shaft (40s).

Shaft (40s) includes first segment (100) and second segment (120). First segment (100) is proximate first lengthwise end (42s) and second segment (120) is proximate second lengthwise end (44s). Within the scope of the current invention, second segment (120) is constructed from rigid biocompatible materials acceptable in the art.

In select preferred embodiments, polyaxial head (150) is connected to non-penetrating margin (46s) of shaft (40s). Polyaxial head (150) of anchor (30) is adapted for connection with an apparatus (not shown) distinct from anchor (30). Examples of apparatus connectable to polyaxial head (150) include but are not limited to: rods, bars, cross-links, screws and locking nuts. Polyaxial head (150) is provided with spheroid (152) connected to non-penetrating margin (46s) of second lengthwise end (44s) of shaft (40s). Prior to surgical fixation, the combination of spheroid (152) and socket (154) allows polyaxial head (150) to be moved in a multitude of axes relative to the longitudinal axis (X-X) of shaft (400. Socket (154) is provided with an outward housing (156) and inward receptacle (158) including one or more threads (160). Selected preferred embodiments of housing (156) can be provided with openings (162, 164, 166) adapted to receive one or more apparatus distinct from anchor (30).

First segment (100) includes at least two divisions (102, 104). As shown in FIG. 6, first segment (100) was cooled below its transition temperature resulting in a second contour or uncurled first segment (100). Generally, second contour of anchor (30) can be utilized by the surgeon for insertion into bone. For example, first segment (100) can be inserted in the lateral mass of the posterior cervical spine. After implantation, when first segment (100) is warmed above the transition temperature, first segment (100) returns to its first contour or curled first segment (100) including divisions (102, 104).

As shown in FIG. 6, tips (108, 110) of transformed divisions (102, 104) have been directed away from longitudinal center of shaft (40s) and directed toward second lengthwise end (44s) of shaft (40s) creating transformed divisions (102, 104) with hook-like contours or curls. Within the scope of the current invention, it is understood that divisions (102, 104) of first segment (100) can also be created with transformed divisions (102, 104) other than hook-like contours.

When first segment (100) returns to its first configuration inside the lateral mass, divisions (102, 104) of first segment (100) can impede movements of the anchor (30) and assist in preventing back out or pull out of anchor (30) from the surgically created cavity. Among other things, in operation, this surgical configuration can clamp a greater volume of cortical bone between curled divisions (102, 104) and polyaxial head (150) of anchor (30) than is possible with current lateral mass fixation screws.

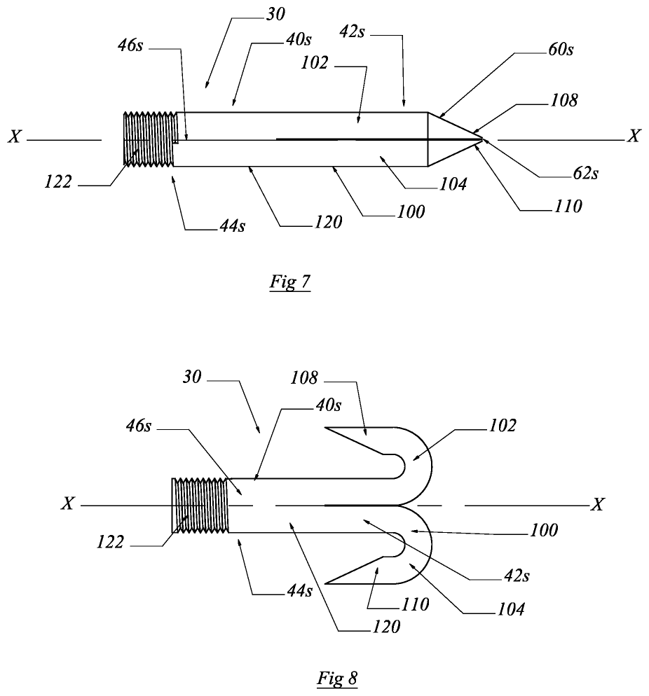

The embodiments, enabled in FIGS. 7 and 8, function similarly to the embodiments represented by FIGS. 5 and 6. However, the embodiments of FIGS. 7 and 8 embodiments do not utilize polyaxial head (150). Instead, second segment (120) of shaft (40s) is provided with at least one thread (122) for connection with an apparatus distinct from anchor (30). Thread (122) can engage an apparatus distinct from anchor (30) such as a nut (not shown).

As used in this Application, “transition temperature” means the temperature at which a portion of the anchor (30) changes from a first contour to a second contour.

Transition temperature ranges for the change from a first distinct shape or contour to a second distinct shape or contour of the temperature sensitive composition of anchor (30) can range from about −20 degrees Celsius to about +110 degrees Celsius. Depending on the transition metals incorporated into the temperature sensitive composition, the range for transition of the current temperature sensitive composition from a first contour to a second contour can range from about −20 degrees Celsius to about +40 degrees Celsius. Select preferred embodiments of the current invention can have transition temperature of from about 20 degrees Celsius to about 33 degrees Celsius. Still other preferred embodiments can have a transition temperature of less than about 35 degrees Celsius. Along with shape memory alloys and shape memory polymers, preferred embodiments of the anchor can be provided with surface treatments in anticipation of improving attachment of bone to the bone fastener or anchor.

The Nonsurgical Preferred Embodiments

The present invention is an anchor that is insertable into a ceiling, floor, roof or wall of a structure. A portion of the anchor is insertable through ceiling, floor, roof or wall of a structure. Depending on the composition into which the anchor will be inserted, the current invention can be self-tapping for select compositions. Due to the anchor's unique construction, the anchor can be used to support a load for generally horizontal surfaces such as ceilings, floors and roofs and generally vertical surfaces such as walls. It is believed that the current anchor can support a load of up to about 35 kilograms when the anchor is inserted into a generally vertical surface and a load of up to about 9 kilograms when the anchor is inserted into a generally horizontal surface. With respect to maximum load and ease of placement, it is believed that the present anchor can provide the benefits of currently available ribbed plastic anchors, self-drilling anchors, molly bolts and toggle bolts while eliminating the shortcomings of currently available anchors.

Preferred embodiments of the present invention are manufactured of metallic alloys, stainless steel, polymers any other composition acceptable in the art. Within the scope of the present invention, it has advantageously been discovered that shafts (40F, 40S) can have lengths from about 50 to about 200 millimeters and heads (150) can have lengths of from about 3 millimeters to about 6.5 millimeters.

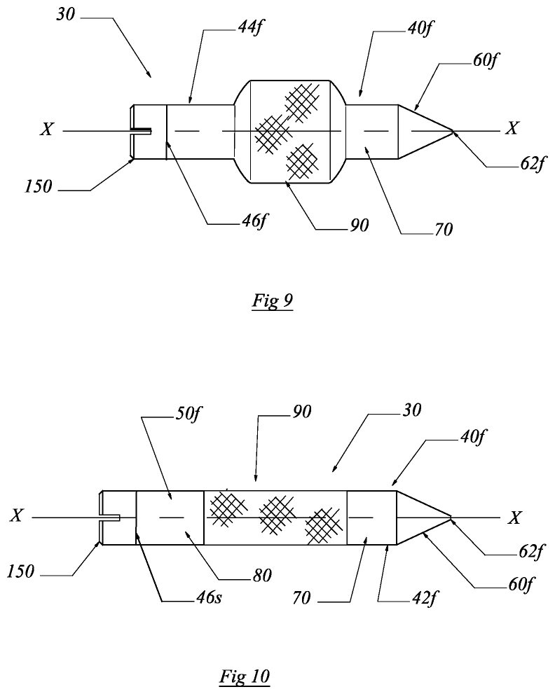

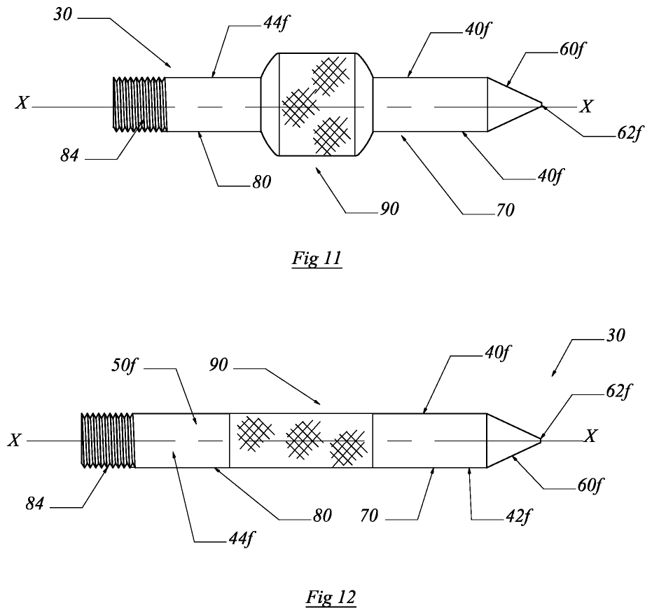

FIGS. 9 and 10 are perspectives of a first preferred embodiment of anchor (30). A preferred embodiment of anchor (30), enabled in FIGS. 9 and 10, includes shaft (400 and head (150). Shaft (400 includes first lengthwise end (420 that is provided with penetrating edge (600. In select preferred embodiments, penetrating edge (600 converges to point (620. Opposite penetrating edge (600 of anchor (30) is non-penetrating margin (460 of second lengthwise end (440 of shaft (400.

Shaft (400 includes first section (70), second section (80) and intermediate section (90). First section (70) is proximate first lengthwise end (420 and second section (80) is proximate second lengthwise end (440. Within the scope of the current invention, first section (70) and second section (80) are constructed from rigid materials acceptable in the art.

Intermediate section (90) of shaft (400 is positioned between first section (70) and second section (80). Intermediate section (90) is constructed of a temperature sensitive composition that can include shape memory metals, shape memory polymers or a combination thereof. The temperature sensitive composition's shape is a first contour above the transition temperature for the temperature sensitive composition and a second configuration below the transition temperature of the temperature sensitive composition. By way of illustration, when intermediate section (90) is cooled below the transition temperature, the second temporary contour of anchor (30) allows insertion of anchor (30) into a generally vertical or horizontal surface. After placement into the ceiling, floor, roof or wall of a structure, when anchor (30) warms above the transition temperature, anchor (30) returns to the first contour.

In select preferred embodiments, intermediate section (90) constructed as a mesh. Intermediate section (90) can include one or more transition metals such as a shape memory alloy. In some preferred embodiments, the shape memory alloy is an alloy of nickel and titanium with percentages of nickel selected as a matter of predetermined engineering parameters.

As shown in FIG. 9, intermediate section (90) is expanded and is above its transition temperature, e.g., −20 degrees Celsius to about 0 degrees Celsius. Expanded intermediate section (90) is adapted to engage a portion of a ceiling, floor, roof or wall into which anchor (30) is inserted.

As shown in FIG. 10, intermediate section (90) was cooled below its transition temperature resulting in a second unexpanded contour for intermediate section (90). Generally, second contour of anchor (30) can be utilized for insertion into a vertical or horizontal surface. For example, with or without the use of a drill to create a hole, first section (70) and intermediate section (90) can be inserted into a portion of a ceiling, floor, roof or wall. After insertion into the vertical or horizontal surface, when intermediate section (90) is warmed above the transition temperature, intermediate section (90) returns to its first expanded configuration. When intermediate section (90) returns to its first configuration inside ceiling, floor, roof or wall, intermediate section (90) can assist in preventing back out or pull out of anchor (30) from the vertical or horizontal surface. In operation, this first configuration anchor (30) contacts a greater surface area of the ceiling, floor, roof or wall than is possible with currently available anchors. It is believed that: the greater surface area of anchor (30) contacting the vertical or horizontal surface of the structure, the greater the resistance to pull out when anchor (30) carries a load.

In select preferred embodiments, head (150) is connected to non-penetrating margin (460 of shaft (400. As shown in FIG. 17, head (150) of anchor (30) can be provided with a receptacle such as a slot, a Phillips, an Allen head, a socket or any other configuration acceptable in the art. Receptacles are adapted to receive such devices as drills and screw drivers. However, unlike many traditional anchors, anchor (30) can be inserted into a vertical or horizontal surface without the aid of a screw driver or drill.

The embodiments, enabled in FIGS. 11 and 12, function similarly to the embodiments represented by FIGS. 9 and 10. The FIGS. 11 and 12 embodiments do not utilize head (150). Instead, second section (80) of shaft (400 is provided with at least one thread (84) for connection with an apparatus distinct from anchor (30) such as a load, a nut or both.

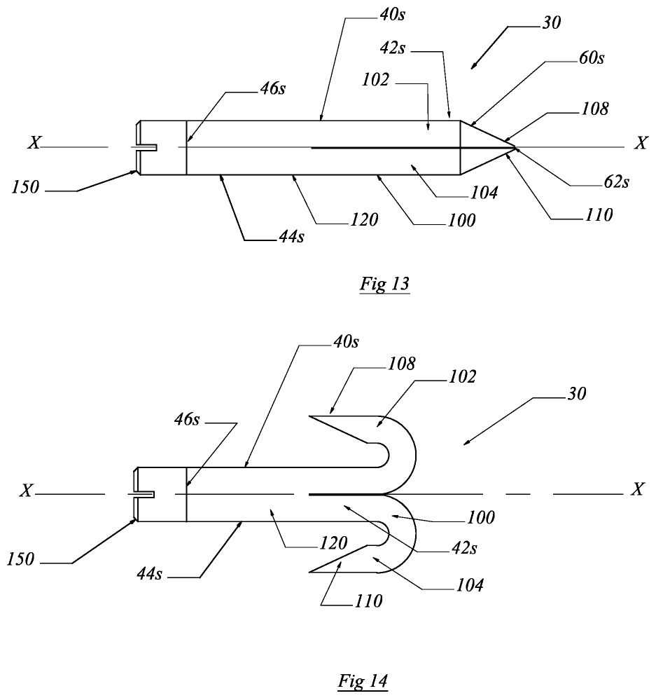

Another preferred embodiment of anchor (30), enabled in FIGS. 13 and 14, includes shaft (40s) and head (150). Shaft (40s) includes first lengthwise end (42s) that is provided with penetrating edge (60s). In select preferred embodiments, penetrating edge (60s) converges to point (62s). Opposite penetrating edge (60s) of anchor (30) is non-penetrating margin (46s) of second lengthwise end (44s) of shaft (40s).

Shaft (40s) includes first segment (100) and second segment (120). First segment (100) is proximate first lengthwise end (42s) and second segment (120) is proximate second lengthwise end (44s). Within the scope of the current invention, second segment (120) is constructed from rigid materials acceptable in the art.

In select preferred embodiments, head (150) is connected to non-penetrating margin (46s) of shaft (40s). Head (150) of anchor (30) is adapted for connection with an apparatus (not shown) such as a load distinct from anchor (30).

First segment (100) includes at least two divisions (102, 104). As shown in FIG. 13, first segment (100) was cooled below its transition temperature resulting in a second contour or uncurled first segment (100). After insertion into a horizontal or vertical surface, when first segment (100) is warmed above the transition temperature, first segment (100) returns to its first contour or curled first segment (100) including divisions (102, 104).

As shown in FIG. 14, tips (108, 110) of transformed divisions (102, 104) have been directed away from longitudinal center of shaft (40s) and directed toward second lengthwise end (44s) of shaft (40s) creating transformed divisions (102, 104) with hook-like contours or curls. Within the scope of the current invention, it is understood that divisions of first segment (100) can also be created with transformed divisions (102, 104) other than hook-like contours.

When first segment (100) returns to its first configuration inside the ceiling, floor, roof or wall, divisions (102, 104) of first segment (100) can impede movements of the anchor (30) and assist in preventing back out or pull out of anchor (30) from the horizontal or vertical surface. In operation, this first configuration anchor (30) contacts a greater surface area of the ceiling, floor, roof or wall than is possible with currently available anchors. It is believed that: the greater surface area of anchor (30) contacting the vertical or horizontal surface of the structure, the greater the resistance to pull out when anchor (30) carries a load.

As shown in FIG. 17, head (150) of anchor (30) can be provided with a receptacle such as a slot, a Phillips, an Allen head, a socket or any other configuration acceptable in the art. In other preferred embodiments of anchor (30), a receptacle such as a slot, a Phillips, an Allen head, a socket or any other configuration acceptable can be incorporated into non-penetrating margin (46f) of shaft (400. Receptacles are adapted to receive such devices as drills and screw drivers. However, unlike traditional wall anchors, anchor (30) can be inserted into a vertical or horizontal surface without the aid of a screw driver or drill.

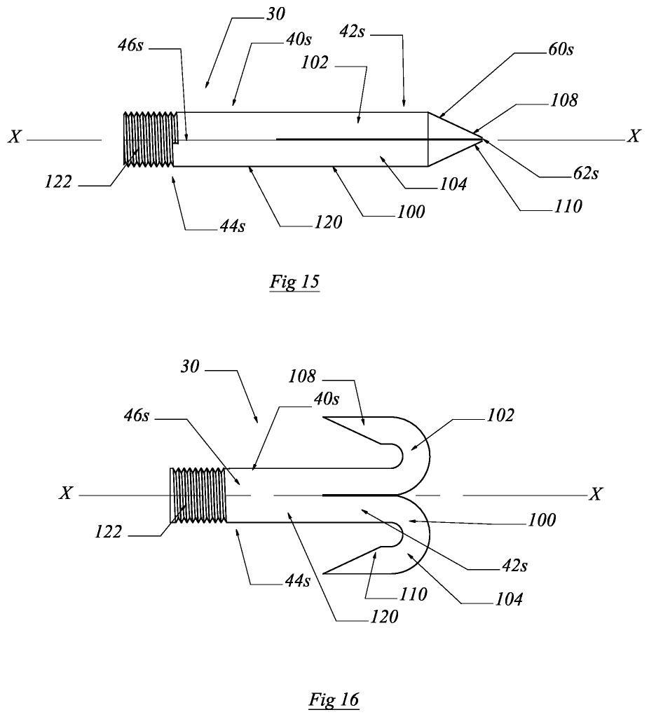

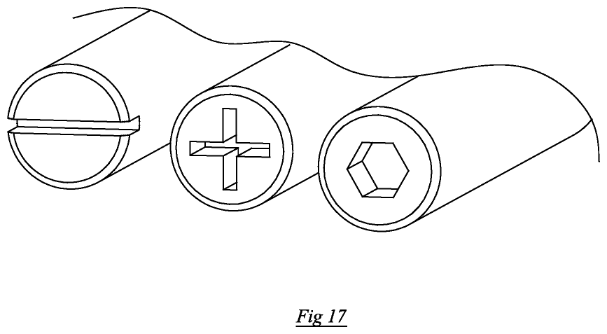

The embodiments, enabled in FIGS. 15 and 16, function similarly to the embodiments represented by FIGS. 13 and 14. The FIGS. 15 and 16 embodiments do not utilize head (150). Instead, second segment (120) of shaft (40s) is provided with at least one thread (122) for connection with an apparatus distinct from anchor (30). Thread (122) can engage an apparatus distinct from anchor (30) such as a load, a nut or both.

As used in this Application, “transition temperature” means the temperature at which a portion of the anchor (30) changes from a first contour to a second contour.

Transition temperature ranges for the change from a first distinct shape or contour to a second distinct shape or contour of the temperature sensitive composition of anchor (30) can range from about −20 degrees Celsius to about +110 degrees Celsius. Depending on the shape memory alloys or shape memory polymers incorporated into the temperature sensitive composition, the range for transition of the current temperature sensitive composition from a first contour to a second contour is from about −20 degrees Celsius to about 0 degrees Celsius. For many uses of the current invention, at ambient temperature, anchor (30) will assume the shape that most resistant to back-out or pull-out

Applicant has enabled, described and disclosed the invention as required by Title 35 of the United States Code.

What is claimed is:

1. An anchor adapted for interconnection with an apparatus distinct from the anchor; the anchor comprising a shaft comprising:
a) a first lengthwise end comprising a conical penetrating edge adapted for insertion into and through a surgically created opening in a bone;
b) a second lengthwise end comprising a non-penetrating margin positioned opposite the conical penetrating edge;
c) a first section proximate the first lengthwise end and a second section proximate the second lengthwise end, wherein the first and second sections are rigid constructions; and
d) a flexible mesh comprising:
i) a first circumference connected to an inward side of the first lengthwise end and a second circumference connected to an inward side of the second lengthwise end; and
ii) a plurality of interwoven shape memory wires or threads or a combination thereof connected between the first circumference and the second circumference; the plurality of interwoven shape memory wires or fibers creating apertures ranging in size from about 1 micrometer to about 5 millimeters; the sizes of the apertures adapted to improve passage of the flexible mesh through the opening, wherein prior to insertion through the opening, a breadth of a middle cross-section of the flexible mesh is equal to or less than diameters of the inward side of the first lengthwise end and the inward side of the second lengthwise end, and subsequent to insertion through the opening, the middle cross-section of the flexible mesh expands to at least twice a size of a bore of the opening, thereby preventing pullout of the anchor from the spinal bone.

2. The anchor of claim 1, wherein the transition temperature for the mesh is influenced by the shape memory wires or shape memory polymeric fibers incorporated into the mesh.

3. The anchor of claim 2, wherein the mesh comprises at least one shape memory alloy or shape memory polymer.

4. The anchor of claim 3, wherein the shape memory alloy comprises nickel and titanium and the shape memory polymer comprises at least one polyurethane, epoxy, polyolefin or polyester.

5. The anchor of claim 4, wherein on achieving the transition temperature, the mesh is expanded or contracted.

6. The anchor of claim 5, wherein the transition temperature is from about 20 degrees Celsius to about 33 degrees Celsius.

7. The anchor of claim 5, wherein the transition temperature is less than about 35 degrees Celsius.

8. The anchor of claim 6 comprising:
a) a polyaxial head mounted to the non-penetrating margin; or
b) a thread wound about a portion of the second lengthwise end.

9. The anchor of claim 8 comprising one or more areas of surface treatments.

10. An anchor adapted for interconnection with an apparatus distinct from the anchor; the anchor comprising:
a) a shaft comprising:
i) a first lengthwise end comprising a first segment including two divisions;
ii) a second lengthwise end comprising a rigid second segment and a non-penetrating margin positioned opposite the first segment; the rigid second segment connected to the first segment; and
iii) the two divisions comprising a temperature sensitive composition, wherein the temperature sensitive composition's is a first contour until a transition temperature for the temperature sensitive composition is achieved, thereby transforming the temperature sensitive composition into a second contour.

11. The anchor of claim 10, wherein the transition temperature for the two divisions is influenced by shape memory alloys, shape memory polymeric fibers or a combination thereof incorporated into the two divisions.

12. The anchor of claim 11, wherein the temperature sensitive composition comprises at least one shape memory alloy or at least one shape memory polymer.

13. The anchor of claim 12, wherein the shape memory alloy comprises nickel and titanium and the shape memory polymer comprises at least one polyurethane, epoxy, polyolefin or polyester.

14. The anchor of claim 13, wherein on achieving the transition temperature, the two divisions are either uncurled or curled.

15. The anchor of claim 14, wherein the uncurled divisions converge to a point.

16. The anchor of claim 14, wherein the curled divisions create a hook-like contour configured to impede movements of the anchor.

17. The anchor of claim 14, wherein the transition temperature is from about 20 degrees Celsius to about 33 degrees Celsius.

18. The anchor of claim 17 comprising:
a) a polyaxial head mounted to the non-penetrating margin; or
b) a thread wound about a portion of the second lengthwise end.

19. The anchor of claim 18 comprising one or more areas of surface treatments adapted to enhance bone ingrowth.

20. An anchor insertable into a ceiling, floor, roof or wall of a structure; the anchor comprising a shaft comprising:
a) a first lengthwise end comprising a penetrating edge adapted to penetrate the ceiling, floor, roof or wall of the structure;
b) a second lengthwise end comprising a non-penetrating margin positioned opposite the penetrating edge;
c) a first section proximate the first lengthwise end and a second section proximate the second lengthwise end, wherein the first and second sections are rigid constructions; and
d) an intermediate section positioned between the first section and the second section; the intermediate section comprising a temperature sensitive composition, wherein the temperature sensitive composition is a first contour until a transition temperature for the temperature sensitive composition is achieved, thereby transforming the temperature sensitive composition into a second contour.
